(19) United States
(12) Patent Application Publication
(10) Pub. No.: US 2012/0213860 A1
(43) Pub. Date: Aug. 23, 2012

(54) XENON-BASED INHALABLE DRUG FOR TREATING OR PREVENTING INDUCED DYSKINESIA

(75) Inventor: Baptiste Bessiere, Issy Les Moulineaux (FR)

(73) Assignee: L'Air Liquide Societe Anonyme Pour L'Etude Et L'Exploitation Des Procedes Georges Claude, Paris (FR)

(21) Appl. No.: 13/502,389

(22) PCT Filed: Nov. 3, 2010
(86) PCT No.: PCT/FR2010/052350

§ 371 Date: Apr. 17, 2012

(30) Foreign Application Priority Data
Nov. 10, 2009 (FR) 0957921

(51) Int. Cl. A61K 33/00; A61P 25/16

(52) U.S. Cl. 424/600

(57) ABSTRACT

The invention relates to the use of a gaseous drug based on gaseous xenon for inhalation for treating or preventing in mammals dyskinesia induced by a dopaminergic agonist. The invention is particularly useful for treating levodopa induced dyskinesia in patients being treated for Parkinson's disease.

The invention relates to a gaseous drug based on gaseous xenon, which can, after inhalation, prevent or treat dyskinesia in mammals, in particular humans, induced by a dopaminergic agonist used in the treatment of Parkinson's disease.

Dyskinesias are disorders that are manifested by abnormal involuntary movements. There are several types, notably:
choreas: irregular, rapid and abrupt movements that affect the face, arms, legs or trunk, for example Huntington's chorea,
ballisms: movements similar to those of the choreas but more jerky and violent,
dystonias: intense muscular contractions usually producing twisting movements and repetitive movements and abnormal positions or postures, and
athetoses: involuntary slow, sinuous and uncoordinated movements of the limbs and especially of the hands, trunk or face.

The movements and other disorders are due to dysfunction of the basal ganglia and of the associated brain structures. Such disorders may occur as a result of hereditary or acquired diseases, idiopathic neurodegeneration or they may be iatrogenic.

The spectrum of disorders is therefore very broad and comprises not only those connected with the power of the movements (akinesia, hypokinesia, bradykinesia) and hypertonia (e.g. Parkinson's disease, certain forms of dystonia, etc.) but also disorders of involuntary movements (hyperkinesias, dyskinesias, etc.).

Knowledge of the pathophysiological mechanisms connected with these disorders suggests that similar mechanisms are mediators of disorders characterized either by hyperkinesias, or by dyskinesias. In other words, treatments that are effective against one form of dyskinesia are also likely to be effective against other forms of dyskinesias having a different etiology. However, this has yet to be demonstrated.

In fact, as noted in the document of O. Rascol et al., Dyskinesia: L-dopa-induced and tardive dyskinesia; Clin. Neuropharmacol.; 2001; November-December; 24(6):313-23, there are two main types of dyskinesias, namely tardive dyskinesias induced by neuroleptics and dyskinesias induced by L-dopa or levodopa, both of which lead to involuntary abnormal movements in those affected.

However, the similarities stop there, since this document clearly states that these two types of dyskinesias are totally different with regard not only to the compounds that induce them but also the underlying diseases.

Regarding the dyskinesias induced by L-dopa or other similar dopaminergic agonists, these dyskinesias appear as a side effect of dopamine replacement therapies employed against the disorders of movement associated with the basal ganglia or Parkinson's syndrome, as described in the document: Molecular mechanisms of L-DOPA-induced dyskinesia, Peter J., Nature Reviews Neuroscience, 2008.

In fact, the symptoms of Parkinson's syndrome are characterized by slow movements (i.e. bradykinesias), rigidity and tremors.

In Parkinson's disease, the primary pathology is a degeneration of the dopaminergic neurons of the substantia nigra, pars compacta.

The currently most widely used symptomatic treatment of Parkinson's syndrome is based on the use of dopamine replacement drugs, e.g. dopamine receptor agonists, in particular levodopa, usually called L-DOPA.

However, these have drawbacks, especially in long-term treatments, as their use can notably produce an erosion of the antiparkinsonian effect of the treatment and the appearance of side effects that are reflected in induced dyskinesias, such as choreas and dystonias.

In this case, the dyskinesias are therefore side effects of treatments of Parkinson's disease by means of L-dopa or other similar dopaminergic agonists.

Now, these side effects limit the use and the benefit of these dopaminergic treatments.

Moreover, the other cause of dyskinesias is treatment of psychoses with neuroleptic drugs, which cause neuroleptic-induced dyskinesias, known under the name of tardive dyskinesias.

To date, a great many treatments of dyskinesias have been proposed but these proved not to be truly effective or they gave rise to undesirable side effects.

Thus, documents WO-A-2005/011711 and U.S. Pat. No. 6,559,190 teach the possible use of inhaled xenon for treating or preventing tardive dyskinesias. However, these documents do not deal with the question of the treatment of dyskinesias induced by the use of a dopaminergic agonist, such as levodopa.

The problem that therefore arises is to propose a medicinal product and a treatment that are effective against dyskinesias occurring in mammals, in particular in humans, which result from or are induced by the use of a dopaminergic agonist, such as levodopa, or a dopamine precursor, which is used in the treatment of Parkinson's disease.

In other words, the present invention does not aim to treat Parkinson's disease as such but to alleviate the adverse effects caused by drugs of the dopaminergic agonist type, such as L-Dopa, used in the treatment of Parkinson's disease, which may cause or induce particular dyskinesias in reaction to their use in the treatment of Parkinson's disease.

Thus, the solution of the invention is based on a gaseous drug based on gaseous xenon for use by inhalation for treating or preventing a dyskinesia in mammals, said dyskinesia being induced by a dopaminergic agonist.

Depending on each individual case, the inhalable gaseous drug according to the invention can comprise one or more of the following characteristics:
the dyskinesia is induced by a dopaminergic agonist.
the mammal is a human being, i.e. a man or a woman, including adolescents or any other group of individuals.
gaseous xenon is mixed with a gas containing oxygen, in particular xenon is mixed with air or N₂/O₂ mixture.
it contains a proportion by volume of xenon of at least 5 vol. % and/or less than 50 vol. %, i.e. typically between 5 and 50%, preferably less than or equal to 40%.
it contains a proportion by volume of xenon of at least 10 vol. % and/or less than or equal to 30 vol. %, preferably between 10 and 30%.
xenon is mixed with at least 21 vol. % of oxygen.
xenon is mixed with at least one other compound selected from nitrous oxide (N₂O), argon, helium, neon, krypton, H₂S, CO, NO and nitrogen.
xenon is administered in combination with a dopamine replacement drug selected from the dopaminergic agonists, in particular levodopa.
xenon is administered by inhalation together with at least one dopaminergic agonist used in the treatment of Parkinson's disease.
xenon is administered prior to, simultaneously with and/or subsequent to administration of the dopamine replacement drug. For example, xenon can be administered simultaneously with the dopamine replacement drug, notably in the form of an aerosol or a suspension comprising gaseous xenon and the dopamine replacement drug in liquid or solid form.
the inhalable drug is administered by inhalation over an administration time in the range from some minutes to some hours and/or at a frequency that can reach one to several times per day or per week.

More generally, the gaseous xenon according to the invention is therefore used for the manufacture of an inhalable drug intended for treating or preventing an induced dyskinesia, and said drug can comprise some or all of the aforementioned characteristics.

In other words, according to the invention, gaseous xenon is administered by inhalation to an individual, i.e. a man or a woman, for treating or preventing a dyskinesia in said individual, in particular a dyskinesia resulting from or induced by a dopaminergic agonist, a dopamine precursor or neuroleptic compound acting on the dopaminergic receptors, the dopaminergic agonist preferably being levodopa. Preferably, the inhalable drug is in gaseous form and contains from 5 to 40 vol. % of xenon and of oxygen.

The present invention is therefore based on a treatment of dyskinesias based on action on the N-methyl-D-aspartate (NMDA) receptors. In fact, the NMDA receptors are a class of excitatory amino acid receptors having many important functions in the motor circuit of the basal ganglia. Thus, the document: Rationale for and use of NMDA receptor antagonists in Parkinson's disease, Hallett P. et al. Pharmacology & Therapeutics, 102 (155-174), 2004, envisages the utilization of these receptors in the treatment of Parkinson's disease.

In the context of the present invention, these NMDA receptors are also regarded as targets of choice for developing a treatment or prevention of dyskinesias induced by a dopaminergic agonist, and moreover for reducing the complications and other side effects caused by therapies for replacement of dopamine, or of neuroleptics.

In fact, the possible role of the NMDA receptors in dyskinesias is supported by the action of NMDA receptor antagonists, for example dextrophan and dextromethorman, in humans and a range of NMDA receptor antagonists in primates treated with MPTP (1-methyl-4-phenyl-1,2,3,6-tetrahydropyridine), which is a powerful selective neurotoxic of dopaminergic neurons (animal model of Parkinson's disease), as shown in the document Molecular mechanisms of L-DOPA-induced dyskinesia, Peter J., Nature Reviews Neuroscience, 2008), since these substances significantly reduce involuntary movements (dyskinesias), but also cause appreciable side effects.

In order to demonstrate the efficacy of xenon in the treatment, i.e. reduction, of dyskinesias induced by a dopaminergic agonist, the following experimental protocol was used, which is based on induction of a dyskinesia by levodopa in an animal model of Parkinson's disease. This model, well accepted in literature, is used conventionally in order to identify future treatments against dyskinesias.

The test results are illustrated in the appended drawings, where:

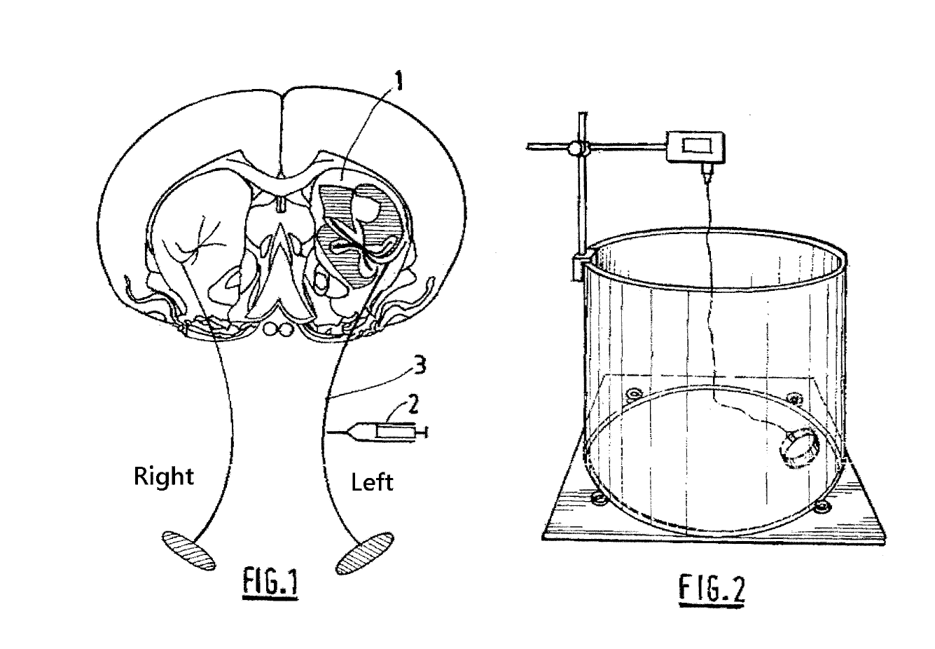

FIG. 1 is a schematic representation of the sites of injection in the brains of rats.

FIG. 2 shows an automatic rotometer used for verifying that the animals are indeed parkinsonian.

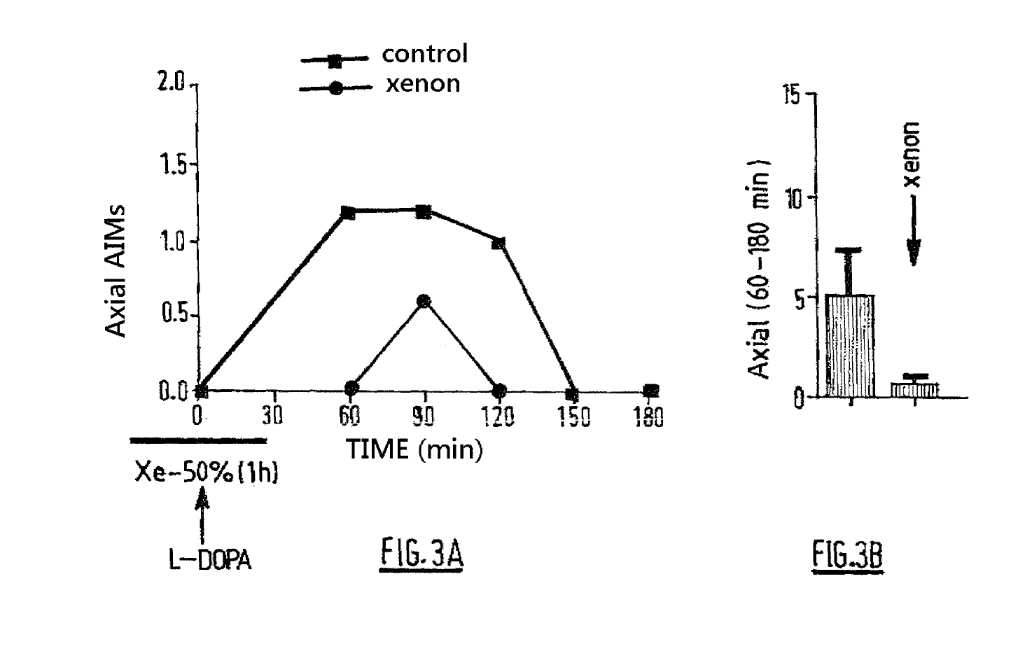

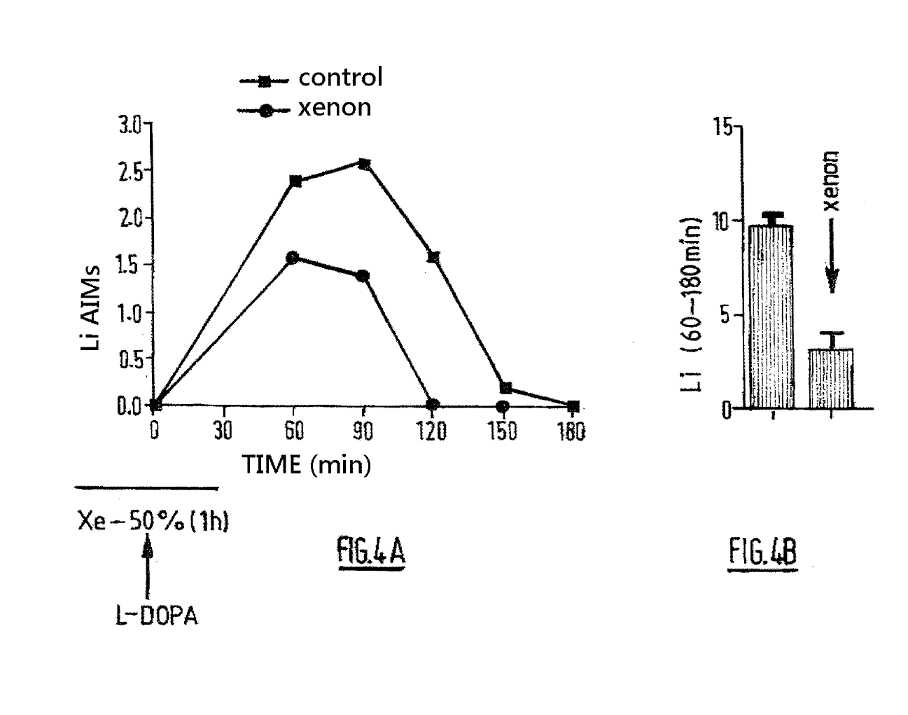

FIGS. 3A and 3B illustrate the effects of xenon on axial dyskinesias in the parkinsonian rat.

FIGS. 4A and 4B illustrate the effects of xenon on hind limb dyskinesias in the parkinsonian rat.

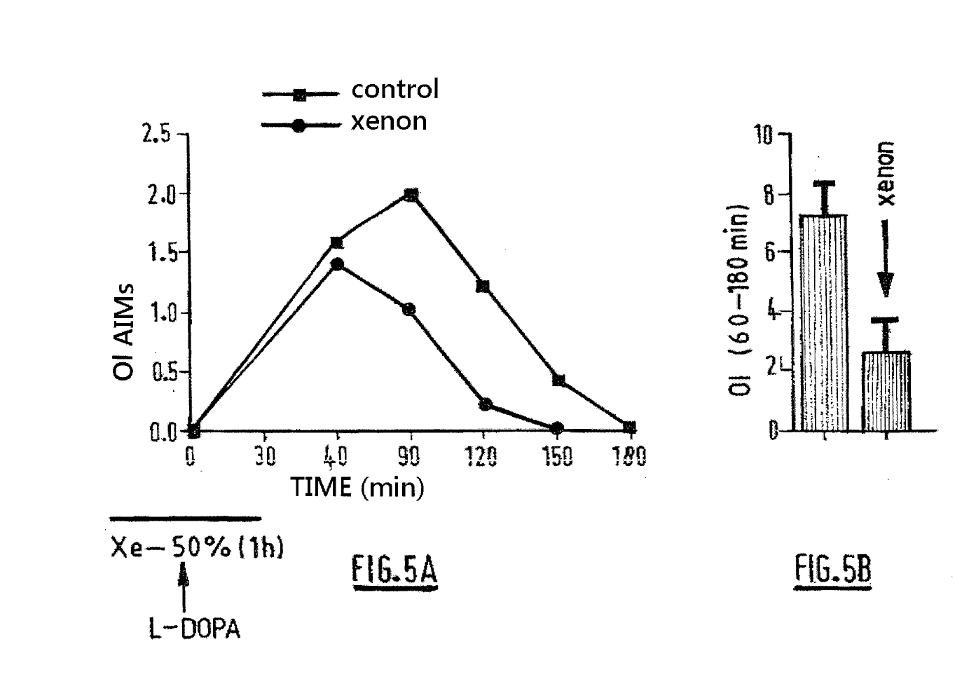

FIGS. 5A and 5B illustrate the effects of xenon on orolingual dyskinesias in the parkinsonian rat.

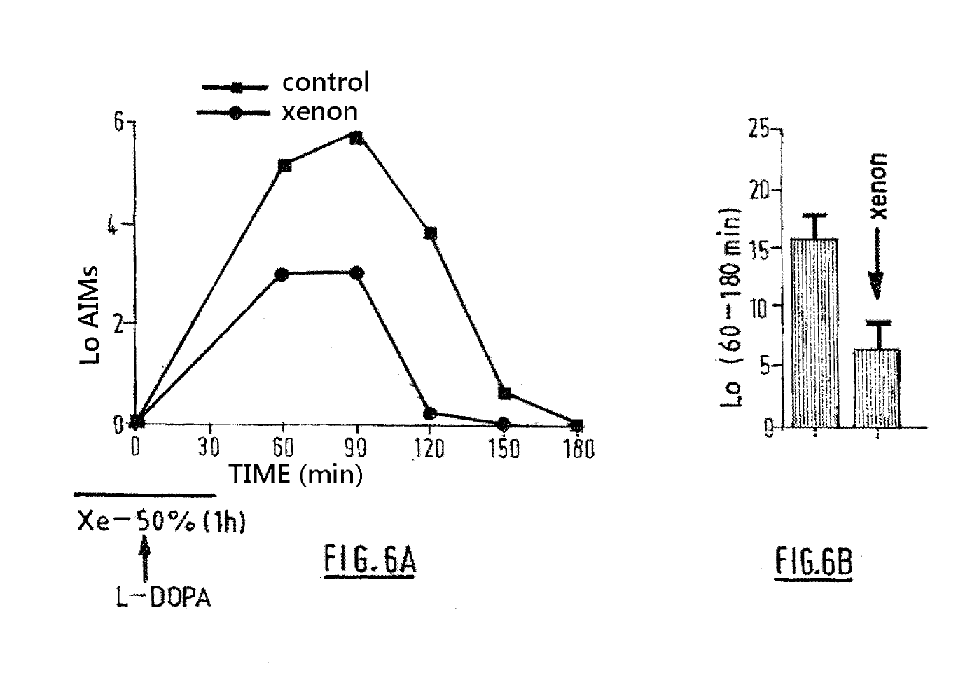

and FIGS. 6A and 6B illustrate the effects of xenon on locomotor dyskinesias in the parkinsonian rat.

More precisely, the effects of administering several gases as treatment of dyskinesias were evaluated in rats having lesions produced by unilateral injection of 6-OHDA (6-hydroxydopamine, a powerful neurotoxic) in an ascending nigrostriatal pathway (animal model of Parkinson's disease).

In this model, long-term treatment of rats with levodopa induces abnormal involuntary movements (AIMs) that are similar to those in dyskinesias induced by levodopa in humans.

The aim of the tests is therefore to demonstrate that inhaled xenon makes it possible to suppress or at least limit the appearance of these AIMs resulting from or induced by levodopa injected in rats, i.e. induced by the molecule used for antiparkinsonian treatment of these rats.

### Experimental Protocol and Results

The rats with lesions induced by 6-OHDA serving as the model of Parkinson's disease are prepared as follows.

16 Sprague-Dawley rats weighing between 250 and 300 g are kept in a room with controlled temperature (about 22° C.) with day/night cycles of 12 h, with free access to food and water. The animals receive, 30 min before the intervention, an intraperitoneal injection of pargyline (5 mg/kg; inhibitor of monoamine oxidase-B) and of desipramine (25 mg/kg; norepinephrine reuptake inhibitor).

The animals are put in an anesthesia chamber with continuous flow of oxygen at 1.5 l/minute and 3% isoflurane. After anesthesia, the animals are placed in a stereotaxic frame.

As can be seen in FIG. 1, 6-hydroxydopamine (6-OHDA) with 0.1% of ascorbic acid (5 mg/ml) dissolved in sterile water is injected manually (ascending nigrostriatal pathway 3 in FIG. 1) using a 5-μl Hamilton 2 syringe in the median fascicle of the right forebrain (at 1) at 5 μl/minute, in the space of 5 minutes. The injection site is localized according to the coordinates relative to bregma: −2.8 mm rostral, 2 mm lateral and 9 mm under the cranium (Paxinos and Watson, 1986). The rats are left to recover for 3 weeks after the lesions.

The animals receive 0.05 mg/kg s.c. of apomorphine. Animals that do not perform 50 complete rotations on the automatic rotometer (see FIG. 2) in the subsequent hour are withdrawn from the study.

The animals that remain receive an administration of 6 mg/kg of levodopa and 15 mg/kg of benzerazide (i.p.), twice a day, for 21 consecutive days. During these 21 days, the abnormal involuntary movements (AIMs) are assessed on days 1, 7, 14 and 21.

The therapeutic treatments begin on day 22. They consist of administering the following to the rats by inhalation:
xenon/O₂ gas mixtures (50%/50% by volume) as test gas according to the invention and;
N₂/O₂ gas mixtures (50%/50% by volume) as control.

The gas mixtures are premixtures supplied by Air Liquide™. The gas concentrations are monitored continuously.

Exposure to the gases is effected in a Plexiglas chamber with a length of 42 cm, and width and height of 26 cm. Each time, 4 or 5 rats are put in the chamber and inhale the test gas for 1 h. The chamber is supplied with fresh gas at a flow rate of 4 l/min.

The abnormal involuntary movements (AIMs) are assessed for 180 min after injection of levodopa. More precisely, the animals are put in boxes (22 cm×34 cm×20 cm) and are left there for 15 minutes for them to become accustomed to their environment. Levodopa methyl ester (6 mg/kg, i.p.) is then administered at time intervals of 1 minute between rats. Each rat is observed for 1 minute at intervals of 30 minutes after injection and for a period of 180 min.

Four subtypes of AIMs are assessed, namely:
Locomotor (Lo): increased movements contralateral to the lesion,
Hind limbs (Li): uncontrolled random movements of the hind limbs contralateral to the lesion,
Orolingual (Ol): excessive mastication and jaw movements with tongue protrusion,
Axial (Ax): dystonic postures or choreiform twisting of the neck and of the upper body contralaterally.

The severity of the MIAs is scored from 1 to 4 according to the duration of the MIAs over an observation period of one minute:
0.1=presence of MIAs for less than 30 s
0.2=presence of MIAs for more than 30 s
0.3=presence of MIAs during the minute with interruption by external stimuli
0.4=presence of MIAs during the minute without interruption by external stimuli

The results obtained confirm that the parkinsonian animals receiving L-Dopa develop 4 types of dyskinesias: Lo, Li, Ol, Ax. This can be seen on the curves of the “control” groups breathing the gas mixture without xenon (N₂/O₂ mixture 50%/50%) shown in FIGS. 3 to 6.

In fact, FIGS. 3A and 3B illustrate the effects of xenon on axial dyskinesias in the parkinsonian rat. As can be seen in FIG. 3A, the kinetics of the effect of xenon on axial dyskinesias following administration of L-Dopa (at t=0) shows a clear decrease in these dyskinesias, both in amplitude and in duration, relative to the control group that breathed the gas mixture not containing xenon.

Moreover, in FIG. 3B, the mean value of the axial dyskinesias from 60 to 180 min reveals that, overall, the rats that breathed the mixture containing xenon have fewer axial dyskinesias (Axial).

In their turn, FIGS. 4A and 4B show the effects of xenon on hind limb dyskinesias (Li) in the parkinsonian rat. As can be seen in FIG. 4A, the kinetics of the effect of xenon on hind limb dyskinesias following administration of L-Dopa (at t=0) shows a significant decrease in these dyskinesias, once again both in amplitude and in duration, relative to the control group that inhaled the gas mixture not containing xenon.

In FIG. 4B, the mean value of hind limb dyskinesias (Li) from 60 to 180 min reveals that on the whole the rats that breathed the mixture containing xenon have fewer hind limb dyskinesias.

FIGS. 5A and 5B illustrate the effects of xenon on orolingual dyskinesias (Ol) in the parkinsonian rat. As can be seen in FIG. 5A, the kinetics of the effect of xenon on orolingual dyskinesias (Ol) following administration of L-Dopa (at t=0) shows a clear decrease of these dyskinesias, both in amplitude and in duration, relative to the control group that breathed the gas mixture not containing xenon.

Moreover, in FIG. 5B, the mean value of orolingual dyskinesias (Ol) from 60 to 180 min reveals that, on average, the rats that inhaled the mixture containing xenon according to the invention have fewer orolingual dyskinesias.

Finally, FIGS. 6A and 6B illustrate the effects of xenon on locomotor dyskinesias (Lo) in the parkinsonian rat. As can be seen in FIG. 6A, the kinetics of the effect of xenon on locomotor dyskinesias following administration of L-Dopa (at t=0) shows a large decrease in these dyskinesias, once again both in amplitude and in duration, relative to the control group that breathed the gas mixture not containing xenon.

Once again, the mean value of locomotor dyskinesias from 60 to 180 min reveals, as shown in FIG. 6B, that overall, the rats that breathed the mixture containing xenon have fewer locomotor dyskinesias.

In conclusion, in the group of animals breathing a gas mixture of xenon/O₂ (50%/50% by volume), appearance of the 4 types of dyskinesias is greatly reduced. This reduction can be seen both in the amplitude and in the duration of the dyskinesias. The test results obtained show that xenon is an effective agent against levodopa-induced dyskinesias and that this gas can therefore be used in the context of a method of therapeutic treatment of dyskinesias in humans.

According to the invention, it is therefore proposed to use gaseous xenon administered by inhalation for treating or preventing dyskinesias in mammals, in particular in humans, and more specifically dyskinesias induced by a dopaminergic agonist, preferably levodopa or a dopamine precursor.

However, it should be emphasized once again that the xenon-based inhalable drug according to the invention is not intended for the treatment proper of Parkinson's disease in itself but makes it possible to prevent, minimize or treat the appearance of a dyskinesia that may result from or be induced by a compound used for the treatment of Parkinson's disease, in particular levodopa.

In general, in a treatment in the context of the present invention using a gaseous drug based on gaseous xenon, xenon can be administered in humans by means of a ventilator, a nebulizer or spontaneously with prepackaged bottles, connected to a face mask or nasal mask, or nasal goggles.

The duration of administration will be selected individually as a function of the severity of the dyskinesia affecting the patient in question, for example xenon can be administered for a time from some minutes to some tens of minutes, or even hours, for example less than one hour, and at a frequency that can be up to one or more times per day or per week, for example once a day for 2 weeks.

The efficacy of the treatment can be evaluated by recording the number and/or frequency for example of levodopa-induced dyskinesias, occurring in a human being during a given period and comparing this number with reference values.

The xenon or xenon-based gas mixture is preferably packaged in a gas cylinder under pressure or in liquid form, for example in a bottle of one or more liters (water content) and at a pressure between 2 and 300 bar.

The xenon or xenon-based gas mixture can be in “ready to use” form, for example premixed with oxygen, or it can be mixed on site at the time of use, notably with oxygen and optionally another gaseous compound or in combination with a dopamine replacement drug, said dopamine replacement drug preferably being selected from a dopaminergic agonist, for example levodopa, as explained above.

1-12. (canceled)

13. A method of treatment or preventing levodopa induced dyskinesia in a mammal, the method comprising a step of administering a gaseous xenon drug by inhalation to the mammal with or at risk of having levodopa induced dyskinesia to thereby treat or prevent the levodopa induced dyskinesia.

14. The method of claim 13 wherein the mammal is a human being.

15. The method of claim 13 wherein the levodopa induced dyskinesia results from a treatment of Parkinson's disease by administration of levodopa to the mammal.

16. The method of claim 13 wherein the gaseous xenon drug is mixed with a gas containing oxygen prior to administration to the mammal by inhalation.

17. The method of claim 13 wherein the gaseous xenon drug comprises a proportion by volume of xenon of between 5 and 50%.

18. The method of claim 13 wherein the gaseous xenon drug comprises a proportion by volume of xenon of between 5 and 40%.

19. The method of claim 13 wherein the gaseous xenon drug further comprises at least 21 volume percent oxygen.

20. The method of claim 13 wherein the gaseous xenon drug further comprises least one other compound selected from nitrous oxide, argon, helium, neon, krypton, H₂S, CO, NO and nitrogen.

21. The method of claim 13 wherein the gaseous xenon drug is administered in combination with levodopa.

22. The method of claim 13 wherein the gaseous xenon drug is administered prior to, simultaneously with and/or subsequent to an administration of levodopa.

23. The method of claim 16, wherein the gas containing oxygen is air or a N₂/O₂ mixture.

24. The method of claim 13 wherein the gaseous xenon drug comprises a proportion by volume of xenon of between 10 and 30%.
